(19) United States
(12) Patent Application Publication
(10) Pub. No.: US 2020/0000863 A1
(43) Pub. Date: Jan. 2, 2020

(54) METHOD OF PROCESSING CANNABIS PLANT MATTER

(71) Applicants: RORY CHESLEY PATRICK MILLIKIN, KELOWNA (CA); MATTHEW KENNEDY, WESTMINSTER, CA (US)

(72) Inventors: RORY CHESLEY PATRICK MILLIKIN, KELOWNA (CA); MATTHEW KENNEDY, WESTMINSTER, CA (US)

(21) Appl. No.: 16/128,242

(22) Filed: Sep. 11, 2018

Related U.S. Application Data
(60) Provisional application No. 62/691,592, filed on Jun. 28, 2018.
(60) Provisional application No. 62/692,628, filed on Jun. 29, 2018.
(60) Provisional application No. 62/696,670, filed on Jul. 11, 2018.
(60) Provisional application No. 62/714,077, filed on Aug. 2, 2018.

(51) Int. Cl. A61K 36/185; A61K 9/20

(52) U.S. Cl. CPC A61K 36/185; A61K 9/20; A61K 2236/15; A61K 2236/13; A61K 2236/17

(57) ABSTRACT

A method of processing cannabis plant matter is provided. It is a particular aspect of the invention to utilize cannabis waste from the processing methods and in a sense recycle and use the byproducts to create additional products. The method describes harvesting cannabis plant matter at a predetermined time, processing the cannabis plant matter to create cannabis juice and cannabis pulp, and utilizing the cannabis pulp to create additional products. This eliminates the waste from processing cannabis plant matter solving a potential crisis in the fact that cannabis plant matter having cannabinoids leech into the soil and water table from disposal.

### CROSS-REFERENCE TO RELATED APPLICATIONS

The present application claims priority to U.S. Provisional Application Ser. No. 62/691,592 filed on Jun. 28, 2018 entitled “A method of juicing a product via a hot and/or cold press”, U.S. Provisional Application Ser. No. 62/692,628 filed on Jun. 29, 2018 entitled “A method to extract cannabinoids from products, U.S. Provisional Application Ser. No. 62/696,670 filed on Jul. 11, 2018 entitled “Systems and methods to create cannabis juice; cannabis secondary products from juicing, cannabis waste recycling; creating fertilizer from cannabis or from other cannabinoid-rich plants; remove chlorophyll, terpenes and taste from cannabis; creating cannabutter, transporting cannabis plants, trim & waste, dry & wet pet food and shampoo”, and U.S. Provisional Application Ser. No. 62/714,077 filed on Aug. 2, 2018 entitled “Systems and Methods To Extract Cannabinoids from Cannabis Plant; To Remove Chlorophyll, Terpenes and/or flavor of from Cannabis Plant; to make Cannabis Oils, Butter and Tinctures, and To Recycle Cannabis Waste” the disclosure of which are hereby incorporated in their entirety at least by reference.

### BACKGROUND OF THE INVENTION

### 1. Field of the Invention

The present invention relates to cannabis, and more particular a method of processing cannabis plant matter.

### 2. Description of Related Art

Cannabis as a health supplement has been practiced for over 1,000 years. Traditionally cannabis is heated to release cannabinoids, primary tetrahydrocannabinol (THC) and cannabidiol (CBD) but also emerging important cannabinoids including but not limited to tetrahydrocannabivarin THCV and cannabinol (CBN) amongst others. These cannabinoids are known to have many therapeutic and medical benefits. Additionally, cannabis in its natural form is a nutrient rich supplement comprising essential fatty acids, aminoacids, fibers, enzymes, vitamins, and minerals. Furthermore, cannabis in its natural form comprises tetrahydrocannabinol acid (THCA) and cannabidiol acid (CBDA), plus other cannabinoids including but not limited tetrahydrocannabivarin acid (THCVA), cannabinol acid (CBNA) which are non-psychoactive cannabinoids providing anti-inflammatory, neuroprotective, anti-emetic, appetite suppressant, sleep inducing, anti-proliferative amongst other medicinal properties.

### BRIEF SUMMARY OF THE INVENTION

In one aspect of the invention a method of processing cannabis is provided, comprising steps: (a) harvesting cannabis plant matter at a predetermined time, wherein the harvesting creates cannabis processing waste; (b) drying the cannabis processing waste; and, (c) processing waste to create a cannabis powder.

In one embodiment, a further step is provided of processing the cannabis powder into pill form. In one embodiment, in step (b), the cannabis processing waste is washed and rinsed. In one embodiment, a step wherein the cannabis processing waste is blanched is provided.

In another aspect of the invention, a method of processing cannabis is provided, comprising steps: (a) harvesting cannabis plant matter in the vegetative stage; (b) washing and rinsing the cannabis plant matter; (c) grinding the cannabis plant matter; (d) juicing the ground cannabis plant matter via a juicer, wherein the ground cannabis plant matter is separated into cannabis juice and cannabis pulp; (e) collecting the cannabis juice for consumption; (f) drying the cannabis pulp; and, (g) processing the cannabis pulp to create a cannabis powder.

In one embodiment, a further step is provided of processing the cannabis powder into pill form. In one embodiment, a step wherein the cannabis processing waste is blanched is provided. In another embodiment, the juicer is selected from the group consisting of a cold press juicer, a centrifugal juicer, or a masticating juicer. In one embodiment, in step (a), the cannabis plant matter comprises organically grown leafy cannabis including cannabis leaves and cannabis stems. In one embodiment, the organically grown leafy cannabis is harvested as a single plant. In yet another embodiment, a step of flash freezing the cannabis plant matter after harvesting is provided.

In yet another aspect of the invention, a method of processing cannabis, comprising steps: (a) harvesting cannabis plant matter at a predetermined time, wherein the harvesting creates a first cannabis processing waste; (b) washing and rinsing the cannabis plant matter and the first cannabis waste; (c) adding taste enhancers to the cannabis plant matter; (d) grinding the cannabis plant matter; (e) juicing the ground cannabis plant matter via a juicer, wherein the ground cannabis plant matter is separated into cannabis juice and a second cannabis processing waste; (f) collecting the cannabis juice for consumption; (g) combining the first and second cannabis processing waste into a combined cannabis waste; (h) drying the combined cannabis waste; and, (i) processing the combined cannabis waste to create a cannabis powder.

In one embodiment, a further comprising of processing the cannabis powder into pill form is provided. In one embodiment, wherein the cannabis plant matter and/or combined cannabis processing waste is blanched. In one embodiment, the juicer is selected from the group consisting of a cold press juicer, a centrifugal juicer, a masticating juicer, or triturating juicer. In one embodiment, wherein the juicer is a cold press juicer consisting of a hydraulic press. In another embodiment, the taste enhancers are selected from the group consisting of fruit juice, vegetable juice, nut juice, or a combination thereof. In one embodiment, the cannabis plant matter, combined cannabis waste, and/or cannabis powder is heated for decarboxylation to occur. In yet another embodiment, the cannabis plant matter, combined cannabis waste, and/or cannabis powder is not heated such that decarboxylation does not occur. In one embodiment, the combined cannabis waste or cannabis powder is used to create clothing, construction materials, microchips, conducting materials, paper, biofuel, plastic composites, food additives, fertilizer, skin care products, fabric, textiles, rope, fuel, isochanvre, commercial and domestic animal feed, protein powder, dietary supplements, dietary powders, cleansing powders, and any consumables in the food and beverage sectors.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a method of processing cannabis plant matter according to an embodiment of the present invention.

FIG. 2 is a method of processing cannabis plant matter according to an embodiment of the present invention.

FIGS. 3A-B described a method of processing cannabis plant matter according to an embodiment of the present invention.

FIG. 4 is a diagram illustrating a method of processing cannabis plant matter according to an embodiment of the present invention.

### DETAILED DESCRIPTION OF THE PREFERRED EMBODIMENT

The following description is provided to enable any person skilled in the art to make and use the invention and sets forth the best modes contemplated by the inventor of carrying out their invention. Various modifications, however, will remain readily apparent to those skilled in the art, since the general principles of the present invention have been defined herein to specifically provide a method of processing cannabis plant matter.

The cannabis plant matter comprises many cannabinoids in its natural state, i.e. raw, including but not limited to tetrahydrocannabinol acid (THCA), cannabidiol acid (CBDA), tetrahydrocannabivarin acid (THCVA), cannabinol acid (CBNA), cannabichromne acid (CBCA), and cannabigerolic acid (CBGA) which have many medicinal properties attributed to them. For instance, TCHA and CBDA may be used to reduce inflammation, inhibit cell growth in tumors and cancer cells, and also suppress muscle spasms. Likewise, CBCA and CBGA may be used to kill or slow bacteria growth, treat fungal infections, and release pain. THCVA may suppress appetite and CBNA may induce sleep.

It is a particular aspect of the invention to utilize cannabis waste from standard processing methods and in a sense recycle and use the byproducts from these processing methods in the method of the present invention. Despite the advantages of using the byproducts, it is a particular advantage when using cannabis plant matter. Specifically, landfills are overwhelmed and environmentalists are declaring a potential crisis in the fact that cannabis plant matter having cannabinoids leech into the soil and water table or if incinerated fill the air with cannbinoids. Thus, the present invention provides a solution to this crisis by utilizing the waste and byproducts from other the standard cannabis processing methods. In one embodiment, cannabis waste is defined as cannabis leaves, stems, small flowers that have been removed during processing, e.g. harvest and trimming. This will be described in greater detail below.

Currently, most cannabis is grown with the desired result of dried cannabis flowers, as well known in the art. This is usually a two-step process after the cannabis plant has reached maturity. First, during harvest, large fan leaves, and sometimes small leaves positioned close to the cannabis flowers are removed leaving mostly the flowers from the cannabis plant which is then hung or placed in racks to dry. After drying, the remaining leaves and visible stems are removed via trimming. Thus, the cannabis waste accumulates in each step of the two-step process. Currently, there are processes to utilize the waste from the second step process for hash, oil, cannabutter, or similar products. However, the waste from the first process is not being utilized due to the seemingly low content of cannabinoids. It is a particular advantage, that the present invention utilizes this waste.

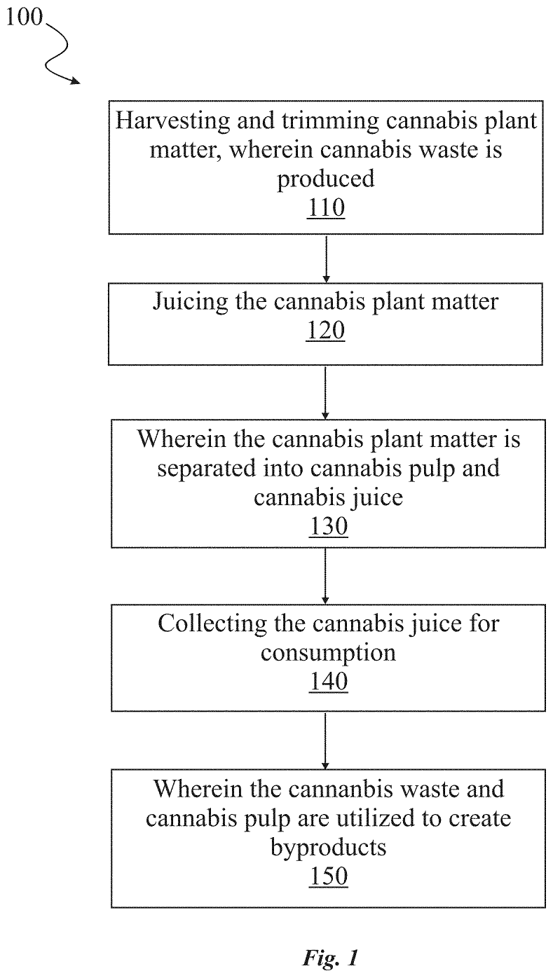

FIG. 1 is a method 100 of processing cannabis plant matter according to an embodiment of the present invention. Referring now to FIG. 1, in step 110, cannabis plant matter is harvested and trimmed, producing cannabis waste as previously described. The harvesting time will be described in greater detail below. In step 120, the cannabis plant matter is juiced. In one embodiment, the juicing is via a cold press juicer, centrifugal juicer, masticating juicer, or triturating juicer. Any commercial cold-press juicer may be used, which relies on a hydraulic press. The details of the cold-press, as well as the specific setting of the hydraulic press, such as press speed and pressure will be discussed in greater detail below. In alternative embodiments, other fruits or vegetables may be juiced with the cannabis matter in step 120, including but not limited to lemons, oranges, pineapples, grapes, mint, cucumbers, bitter blockers, or other taste changers, etc. This is a particular advantage of the present invention, as the flavor profile of the cannabis juice is improved. In step 130, the cannabis is separated into cannabis pulp and cannabis juice. In step 140, the cannabis juice is collected for consumption. In some embodiments, additional products, such as fruits, vegetables, nuts, and/or spices may be combined with the juice to improve flavors and taste, wherein the additional products are preprocessed into juice, puree, or powdered form to mix easily with the cannabis juice. The cannabis juice may be consumed to take advantages of medicinal properties of the plant, including but not limited to anti-inflammatory, neuroprotective, anti-emetic, appetite suppressant, sleep inducing and anti-proliferative properties, as well as the nutrients, essential fatty acids, aminoacids, fibers, enzymes, vitamins, and minerals.

It is a particular advantage of the present invention to utilize the cannabis pulp waste from this process. In step 150, the cannabis pulp may be utilized along cannabis waste obtained from the harvest as described above to create additional products, defined as byproducts. In one embodiment, there are a variety of means to which the byproducts are created. For instance, the cannabis pulp and/or waste may be subjected to various treatments including but not limited to drying, dehydrating, boiling, steaming, cooking, pressing, microwaving, infrared, flash freezing, freezing, blanching, frying, re-thermalizing, and centrifuging to create powder, liquid, mulch, fertilizer, solids and other forms of byproducts for re-purposing useful in a variety of purposes. In one embodiment, the variety of purposes may include but are not limited to clothing, construction materials (ie. insulation, hempcrete (concrete), steel, rebar, drywall, studs, beams, flooring, windows), microchips, conducting materials, paper, biofuel, plastic composites, food additives, fertilizer, skin care products, fabric, textiles, rope, fuel, isochanvre, commercial and domestic animal feed, protein powder, dietary supplements, dietary powders, cleansing powders, and any consumables in the food and beverage sectors. In a preferred embodiment, the cannabis pulp and cannabis waste defined as cannabis process waste is dehydrated, then grinded to create a powder. Although dehydrating is the preferred method, any known method of drying may be used, including natural methods, e.g. sun drying, or machine methods, e.g. heated via a machine. In some embodiments, other methods may be utilized to create the power, including but not limited to crushing, rolling, shaking, centrifuging, smashing, etc. In some embodiments, the powder is capsuled in pill form. In other embodiments, the power is pressed into a hard pill. Yet in other embodiments, the power is liquefied using known methods to create gel caps. In some embodiments, prior to or after grinding the cannabis process waste is blanched, removing the terpenes and/or chlorophyll to improve taste for consumption. In some embodiments, the cannabis plant matter is blanched prior to the juicing method described above. The blanching details will not be described herein, and are discussed in provisional patent application 62/714,077 incorporated by reference.

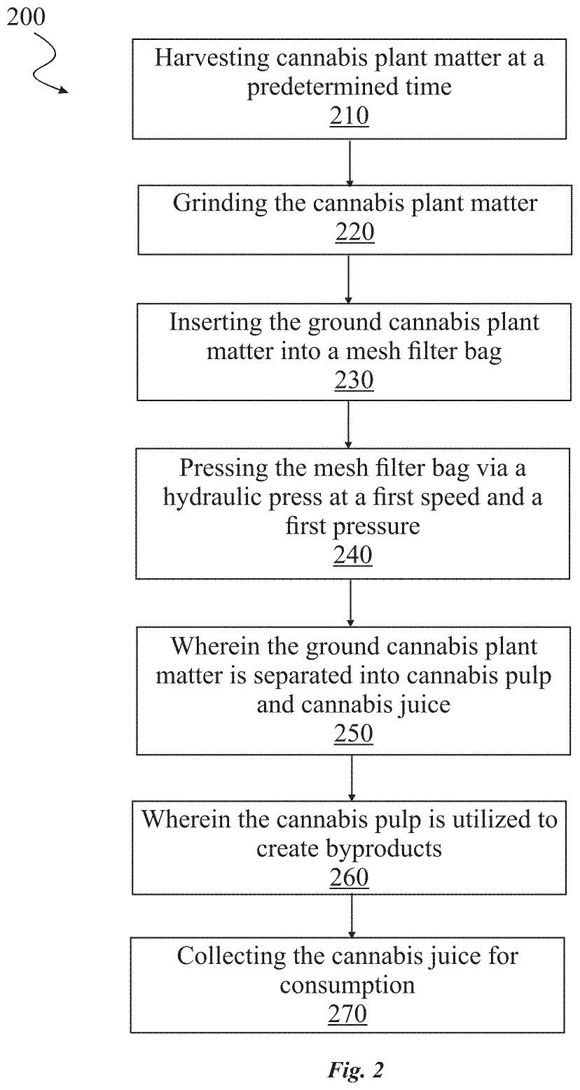

FIG. 2 is a method 200 of processing cannabis plant matter according to an embodiment of the present invention. Referring now to FIG. 2, in step 210, cannabis plant matter is harvested at a predetermined time. In some embodiments, the cannabis plant matter comprises cannabis leaves and stems. Traditionally, cannabis plants are harvested exclusively for their flowers, and cannabis flowers have an ideal maturity date which varies by cannabis variety, including but not limited to Cannabis sativa and cannabis indica. Depending on the variety, the harvesting time may be between 8 to 11 weeks. During this harvesting time, and sometimes a week or two prior, the cannabis leaves start showing signs of nutrient deficiency as the cannabis plant needs all of its available nutrients to support the cannabis flowers. In some embodiments, it is a particular advantage of the present invention to harvest the cannabis plant before the cannabis flowers form, such as in the vegetative state. This allows the whole leafy cannabis plant to be harvested without harvesting the cannabis leaves and stems separately from the flowers. Furthermore, by harvesting the entire leafy cannabis plant it reduces the amount of handling and also minimizes the amount of oxidation that occurs after harvesting, as the cannabis plant matter is exposed to air. Oxidation causes the cannabis plant matter to be subject to nutrient degradation, thus it is a goal to limit oxidation prior to processing. In the preferred embodiment, the cannabis plant matter is harvested during the vegetation stage, prior to the flowering stage allowing for the whole leafy cannabis plant to be harvested prior to flower development. The cannabis plant matter may be either from a male or female cannabis variety. Another particular advantage of harvesting the cannabis plant before the cannabis flowers form is that more cannabis plants may be grown in a shorter amount of time, as the growing cycle is reduced. This allows cannabis plant farmers to maximum crop yields, reduce the possibility of pests, insects, mites, etc. from infecting the cannabis crop. It is also critical that the harvested cannabis plant matter be free of any pesticides and be from organically grown cannabis plants. This ensures the cannabis is safe for consumption.

Still referring to FIG. 2, in step 220, the cannabis plant matter is grinded. To reiterate, the cannabis plant matter in this embodiment is the whole cannabis plant harvested in the vegetative stage as described above. The grinding allows for the maximum amount of plant matter to be processed, preferably via juicing, and more specifically increases the amount of surface area of plant matter increasing efficiency. In some embodiments, the cannabis plant matter is ground using a grinder having a blade length of ¼″. This blade may be of any shape, but is preferably constructed of a circular shape. It is also critical that the grinder is attached to the juicing apparatus to limit handling to prevent possible contamination from the operators. The juicing apparatus will be discussed in greater detail below. In some embodiments, the blade is rotated at a maximum speed of 40 hertz or 2400 revolutions per min (RPM). This speed avoids heating the cannabis plant matter to a point where possible reactions may occur, including but not limited to nutrient degradation and acid cannabinoids being converting to non-acid cannabinoids which may lead to psychoactive effects for the consumer. For instance, during operation if the temperature of the cannabis plant matter reaches a temperature threshold a portion of the tetrahydrocannabinol acid (THCA) and cannabidiol acid (CBDA) may be converted into tetrahydrocannabinol (THC) and cannabidiol (CBD) respectively, i.e. decarboxylation.

In step 230, the ground cannabis plant matter is inserted into a mesh filter bag. In some embodiments, the grinder is a hopper style grinder allowing the ground cannabis plant matter to fall directly into the mesh bag. This is particularly advantageous as it reduces the handling of the fresh plant matter maintaining optimally cleanliness. The mesh filter bag comprises openings having a size of 400 to 600 microns. It should be understood that the openings are not limited to the aforementioned sizes, and optimum sizes may be discovered by routine experimentation. After pressing, which will be discussed in greater detail below, the mesh filter bag is designed to retain the pulp while allowing the juice to pass through the openings. In alternative embodiments, equivalent methods to a mesh bag may be used such as catches and trays, i.e. any method to retain the pulp. Next, in step 240, the mesh filter bag is pressed via a hydraulic press at a speed and a pressure. As previously mentioned, heating the cannabis plant matter to a point where possible reactions may occur, including but not limited to nutrient degradation and acid cannabinoids being converting to non-acid cannabinoids which may lead to psychoactive effects for the consumer is not desired. Consequently, the speed and pressure must not allow the cannabis plant matter to be heating above a threshold temperature where the possible reactions may occur. The pressure increases when the ground cannabis plant matter is actively being pressed between plates operated via the hydraulic press. The maximum pressure is 1800 pounds per square inch (PSI). Pressing at a slow speed and not exceeding the maximum pressure allows for the maximum amount of juice to be extracted without causing possible reactions.

In step 250, the ground cannabis matter is separated in cannabis pulp and cannabis juice, wherein the cannabis pulp (cannabis processing waste) may be utilized to create byproducts as previously described via step 260. In step 270, the cannabis juice is collected for consumption.

In some embodiments, decarboxylation is preferred. Therefore, the maximum pressure in step 240 above may be increased as needed, as well as the grinding speed in step 220. Further, the hydraulic plates may be heated for decarboxylation to occur. Alternatively, if desired, the cannabis juice and/or pulp may be heated for decarboxylation to occur. Likewise, the decarboxylation may occur after the cannabis processing waste has been processed into a powder.

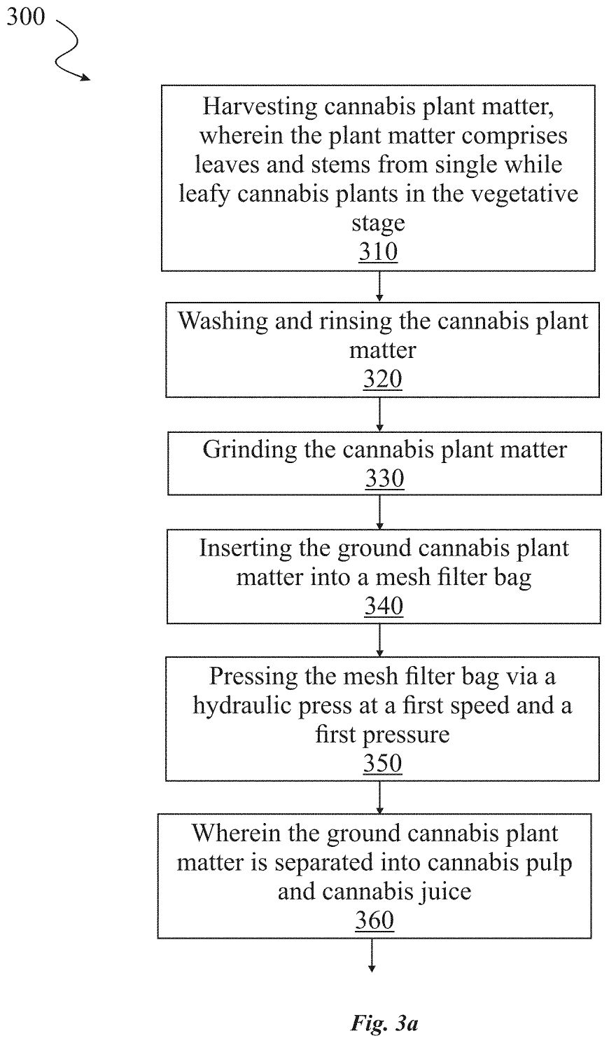

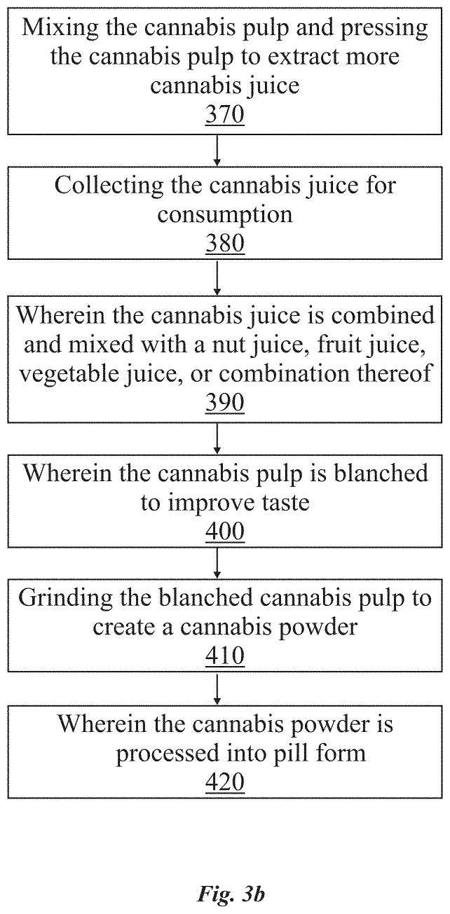

FIG. 3 is a method 300 of processing cannabis plant matter via juicing according to an embodiment of the present invention. Referring now to FIG. 3, in step 310, cannabis plant matter is harvested. In some embodiments, the cannabis plant matter comprises cannabis leaves and stems. In some embodiments, the cannabis plant matter does not include cannabis flowers and ideally the cannabis plant matter is harvested from single whole leafy cannabis plants in the vegetative stage. In step 320, the cannabis plant matter is washed and rinsed prior to juicing. This is an optional step, as the cannabis plant matter and whole leafy cannabis plants should be organically grown, however the cannabis plant matter may be washed and rinsed to remove any potential containments, bugs, dirt, and dust off prior to juicing. Next, in step 330, the cannabis plant matter is grinded. In one embodiment, the grinder is attached to the juicing apparatus to limit handling to prevent possible contamination from the operators. In other embodiments, the grinder is a separate from the juicing apparatus. In one embodiment, the grinding speed should avoid heating the cannabis plant matter to a point where possible reactions may occur, including but not limited to nutrient degradation and acid cannabinoids being converting to non-acid cannabinoids which may lead to psychoactive effects for the consumer. In other embodiments, as previously discussed, decarboxylation is desired, and various methods may be utilized to carry out the decarboxylation, including steps during the juicing or after the juicing.

In step 340, the ground cannabis plant matter is inserted into a mesh filter bag. In some embodiments, the grinder is a hopper style grinder allowing the ground cannabis plant matter to fall directly into the mesh bag. This is particularly advantageous as it reduces the handling of the fresh plant matter maintaining optimally cleanliness. It should be understood, that in alternative embodiments a mesh filter bag is not required if trays and catches are set up to catch the cannabis pulp, however a mesh filter bag is preferred. Next, in step 350, the mesh filter bag is pressed via a hydraulic press at a speed and a pressure.

In step 360, the ground cannabis matter is separated into cannabis pulp and cannabis juice. Next, in step 370, the remaining cannabis pulp is mixed in the mesh filter bag and the mesh filter bag is pressed again to extract additional cannabis juice from the cannabis pulp. Next, in step 380, the cannabis juice is collected for consumption. Although, not noted, any step and method for processing discussed herein may be included in any of the methods disclosed. For instance, in this method, fruits, vegetables, and/or nuts may be juiced with the cannabis matter or combined with the juice if already processed. It is preferred that the combination is done after processing, so that the cannabis processing waste is pure cannabis. For instance, in one embodiment, in step 390, the cannabis juice is combined with a nut juice, fruit juice, vegetable juice, or any combination thereof. However, in alternative embodiments, nut juice, fruit juice, vegetable juice, etc. may be juiced with the cannabis in step 350. Then, in step 400 the cannabis pulp (cannabis processing waste) may be utilized to create byproducts as previously described. For instance, in a preferred embodiment the cannabis pulp is blanched to improve taste. In one embodiment, the cannabis pulp is blanched in a salt, saline, and/or sodium liquid to help remove additional chlorophyll. Next, in step 410, the blanched cannabis pulp is ground to create a cannabis powder. Last, in step 420, the cannabis powder processed into pill form.

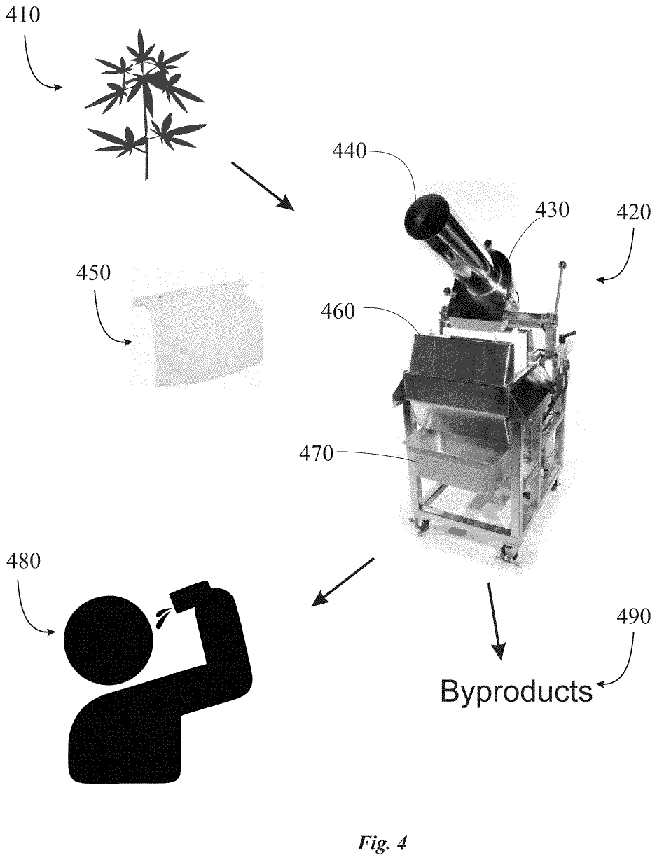

FIG. 4 is a diagram illustrating a method of juicing cannabis plant matter according to an embodiment of the present invention. In some embodiments, organic leafy cannabis plants 410 are grinded by a grinder 430, wherein the grinder is attached to a hydraulic press 420. The leafy cannabis plants and corresponding plant matter are feed into a hopper 440 which guides the plant matter into the grinder at a controlled speed as well known in the art. The ground plant matter is falls directly into a mesh filter bag 450 which is located between a pair of hydraulic press plates 460. The mesh filter bag has been removed from the hydraulic press for clarity. The hydraulic press is then activated, and the pair of hydraulic press plates crushes the ground plant matter located into the mesh filter bag separating the ground plant matter into a pulp and a juice. The pulp remains in the mesh filter bag, while the juice is collected in a container 470. The cannabis juice is then ready for consumption 480. In some embodiments, the cannabis juice is mixed with other juices, including but not limited to nut, vegetable and fruit juices allowing for the cannabis juice to be more appetizing. Byproducts 490 and additional products are created from the pulp.

In some embodiments, depending on where and how the cannabis plant matter is received to carry out the methods described above, the cannabis plant matter and/or cannabis processing waste may be flash frozen, i.e. frozen rapidly to prevent the formation of ice crystals on the cannabis plant matter. In other embodiments, the cannabis plant matter and/or cannabis processing waste may be frozen. The flash frozen or frozen cannabis is then processed via the methods described above.

Although the invention has been described in considerable detail in language specific to structural features and or method acts, it is to be understood that the invention defined in the appended claims is not necessarily limited to the specific features or acts described. Rather, the specific features and acts are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention. For instance, the methods described above may be modified for commercial applications and utilize bath processing, or continuous flow processing methods.

In addition, reference to “first,” “second,” “third,” and etc. members throughout the disclosure (and in particular, claims) are not used to show a serial or numerical limitation but instead are used to distinguish or identify the various members of the group.

What is claimed is:

1. A method of processing cannabis, comprising steps:
(a) harvesting cannabis plant matter at a predetermined time, wherein the harvesting creates cannabis processing waste;
(b) drying the cannabis processing waste; and,
(c) processing waste to create a cannabis powder.

2. The method of claim 1, further comprising a step (d) processing the cannabis powder into pill form.

3. The method of claim 1, wherein in step (b), the cannabis processing waste is washed and rinsed.

4. The method of claim 1, further comprising a step wherein the cannabis processing waste is blanched.

5. A method of processing cannabis, comprising steps:
(a) harvesting cannabis plant matter in the vegetative stage;
(b) washing and rinsing the cannabis plant matter;
(c) grinding the cannabis plant matter;
(d) juicing the ground cannabis plant matter via a juicer, wherein the ground cannabis plant matter is separated into cannabis juice and cannabis pulp;
(e) collecting the cannabis juice for consumption;
(f) drying the cannabis pulp; and,
(g) processing the cannabis pulp to create a cannabis powder.

6. The method of claim 5, further comprising a step (h) processing the cannabis powder into pill form.

7. The method of claim 5, further comprising a step wherein the cannabis plant matter is blanched.

8. The method of claim 5, wherein the juicer is selected from the group consisting of a cold press juicer, a centrifugal juicer, a masticating juicer, or a triturating juicer.

9. The method of claim 5, wherein in step (a), the cannabis plant matter comprises organically grown leafy cannabis including cannabis leaves and cannabis stems.

10. The method of claim 9, wherein the organically grown leafy cannabis is harvested as a single plant.

11. The method of claim 5, further comprising a step of flash freezing the cannabis plant matter after harvesting.

12. A method of processing cannabis, comprising steps:
(a) harvesting cannabis plant matter at a predetermined time, wherein the harvesting creates a first cannabis processing waste;
(b) washing and rinsing the cannabis plant matter and the first cannabis waste;
(c) adding taste enhancers to the cannabis plant matter;
(d) grinding the cannabis plant matter;
(e) juicing the ground cannabis plant matter via a juicer, wherein the ground cannabis plant matter is separated into cannabis juice and a second cannabis processing waste;
(f) collecting the cannabis juice for consumption:
(g) combining the first and second cannabis processing waste into a combined cannabis waste;
(h) drying the combined cannabis waste; and,
(i) processing the combined cannabis waste to create a cannabis powder.

13. The method of claim 12, further comprising a step (j) processing the cannabis powder into pill form.

14. The method of claim 12, further comprising a step wherein the cannabis plant matter and/or combined cannabis processing waste is blanched.

15. The method of claim 12, wherein the juicer is selected from the group consisting of a cold press juicer, a centrifugal juicer, a masticating juicer, or a triturating juicer.

16. The method of claim 12, wherein the juicer is a cold press juicer consisting of a hydraulic press.

17. The method of claim 12, wherein the taste enhancers are selected from the group consisting of fruit juice, vegetable juice, nut juice, bitter blockers, artificial or natural flavors, or a combination thereof.

18. The method of claim 12, wherein the cannabis plant matter, combined cannabis waste, and/or cannabis powder is heated for decarboxylation to occur.

19. The method of claim 12, wherein the cannabis plant matter, combined cannabis waste, and/or cannabis powder is not heated such that decarboxylation does not occur.

20. The method of claim 12, wherein the combined cannabis waste or cannabis powder is used to create clothing, construction materials, microchips, conducting materials, paper, biofuel, plastic composites, food additives, fertilizer, skin care products, fabric, textiles, rope, fuel, isochanvre, commercial and domestic animal feed, protein powder, dietary supplements, dietary powders, cleansing powders, and any consumables in the food and beverage sectors.
